(19) United States
(12) Patent Application Publication
(10) Pub. No.: US 2024/0156487 A1
(43) Pub. Date: May 16, 2024

(54) MEDICANT DELIVERY DEVICE

(71) Applicant: GYRUS ACMI, INC D/B/A OLYMPUS SURGICAL TECHNOLOGIES AMERICA, Westborough, MA (US)

(72) Inventors: Madeline C. Graham, Sammamish, WA (US); Hugo X. Gonzalez, Woodville, WA (US); Jason T. Panzenbeck, Redmond, WA (US)

(21) Appl. No.: 18/417,486

(22) Filed: Jan. 19, 2024

Related U.S. Application Data
(63) Continuation of application No. 17/011,026, filed on Sep. 3, 2020.
Application No. 17/011,026 is a continuation of application No. 16/184,231, filed on Nov. 8, 2018, now Pat. No. 11,116,538.
(60) Provisional application No. 62/895,337, filed on Sep. 3, 2019.

(51) Int. Cl. A61B 17/3205; A61B 1/267; A61B 17/32

(52) U.S. Cl. CPC A61B 17/3205; A61B 1/2676; A61B 17/320016

(57) ABSTRACT

An example medical instrument for creating tracks within target tissue. Medicants could be injected into these tracks for diffusion into the surrounding tissue. An exemplary medical device includes a first tissue penetrating device, a second tissue penetrating device and a handle. The handle includes a first component connected to the first tissue penetrating device and a second component connected to the second tissue penetrating device. When the second component is at a first position relative to the first component, the second tissue penetrating device is in a non-track creation configuration. When the second component is at a second position relative to the first component, the second tissue penetrating device is in a track creation configuration. The second tissue penetrating device is slidably received within the first tissue penetrating device.

### PRIORITY CLAIM

This application is a Continuation of U.S. patent application Ser. No. 17/011,026, filed Sep. 3, 2020, which is a continuation of U.S. application Ser. No. 16/184,231 filed Nov. 8, 2018 and now issued as U.S. Pat. No. 11,116,538, and also claims the benefit of U.S. Provisional Application No. 62/895,337, filed Sep. 3, 2019, the contents of which are hereby incorporated by reference.

### BACKGROUND

There are several medical situations where it is desired to deliver substances by injection to a relatively large volume of tissue. If a substance is delivered by a single point injection, the problems that may arise are (a) the substance cannot spread throughout the volume in sufficient time; (b) too much dilution may occur during the spreading; (c) the distribution of the substance within the volume may be very inhomogeneous; and (d) unwanted spreading to regions away from the target volume may occur. One solution to this problem is to give smaller injections at several sites within the target volume. This approach has at least a few disadvantages: (a) multiple needle stab wounds are created; (b) accuracy of placement is limited; and (c) the time for the procedure may be increased.

### SUMMARY

The embodiments shown relate to an improved hypodermic needle and fluid injection device.

The embodiments shown an example medical instrument for creating tracks within target tissue. Medicants could be injected into these tracks for diffusion into the surrounding tissue.

Further features, advantages, and areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way. The components in the figures are not necessarily to scale, emphasis instead being placed upon illustrating the principles of the embodiments. In the drawings:

FIG. 1 is a side view of external components of an injection system formed in accordance with an embodiment;

FIG. 2 is an x-ray view of a distal end of a component of the injection system of FIG. 1;

FIG. 3 is a side, x-ray view of the distal end of the component of FIG. 2 in a first configuration;

FIG. 4 is a side x-ray view of the distal end of the component of FIG. 2 in a second configuration;

FIG. 5 is a perspective view of a handle associated with the component of FIG. 2;

FIG. 6-1 is an x-ray view of a distal end of an injection component formed in accordance with an embodiment;

FIG. 6-2 is an x-ray view of the injection component of FIG. 6-1 in a different operational state;

FIG. 6-3 is a side view of a stylet device used in the injection component of FIGS. 6-1 and 6-2 having a first pattern of injection;

FIG. 6-4 is a side view of a stylet device used in the injection component of FIGS. 6-1 and 6-2 having a second pattern of injection;

FIG. 7-1 is an x-ray view of the injection component in a first operational state;

FIG. 7-2 is an x-ray view of the injection component of FIG. 7-1 in a second operational state;

FIG. 8-1 is an x-ray view of a distal end of an injection component formed in accordance with an embodiment;

FIG. 8-2 is an x-ray view of the injection component of FIG. 8-1 in a first operational state;

FIG. 9-1 is a partial cross-sectional view of a distal end of an injection component formed in accordance with an embodiment;

FIG. 9-2 is a partial cross-sectional view of the stylet component of FIG. 9-1 in a first operational state; FIG. 10 is a side view of stylet device formed in accordance with an embodiment;

FIG. 11-1 is a side view of a distal end of an injection component formed in accordance with an embodiment;

FIG. 11-2 is side view of a stylet of the injection component of FIG. 11-1; and

FIG. 11-3 is an x-ray view of a distal end of the injection component of FIG. 11-1.

### DETAILED DESCRIPTION

The following description is merely exemplary in nature and is not intended to limit the present disclosure, application, or uses.

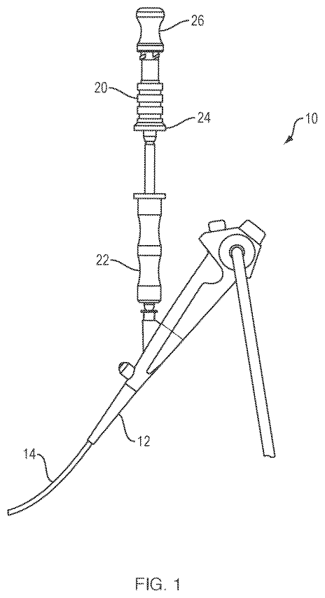

FIG. 1 shows an exemplary system 10 for injecting fluid or medicants into target tissue. The system 10 includes an injection device 20 that is received through a handle 16 and an insertion tube 14 of an endoscope 12 (e.g., bronchoscope). The injection device 20 may or may not be used with a sheath. If the injection device 20 is used with the sheath (not shown), a handle of the injection device 20 includes a base component 22 connected to the sheath, an actuator component 24 connected to a needle device (not shown) slidably received within the sheath and a stylet handle component 26 connected to a stylet (not shown) slidably received within the needle device.

The actuator component 24 allows a user to extend a distal end of the needle distally from a distal end of the sheath. Force applied by the user to the actuator component 24 translates into a force at the distal end of the needle, thus allowing for penetration of the needle into target tissue (e.g., lesion or tumor).

A stylet extension mechanism (shown by example in FIG. 5) allows for extension of at least a portion of the stylet from a distal section of the needle. The stylet extension mechanism may include components located in the actuator component 24 and/or the stylet handle component 26. Examples of the stylet extension mechanism are shown and/or described below.

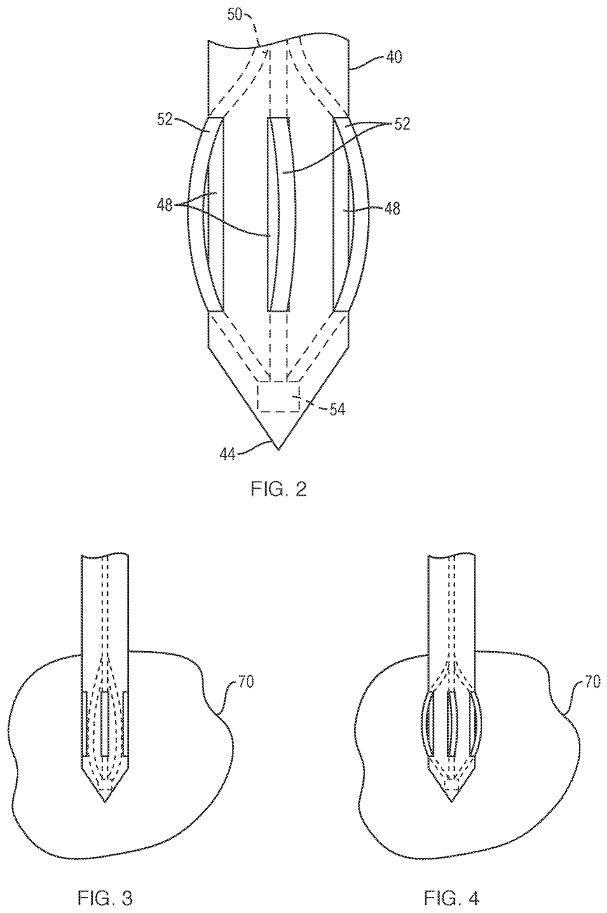

As shown in FIG. 2, a needle 40 of the injection device 20 (FIG. 1) includes a trocar or occluded tip 44. The needle 40 includes one or more slots 48 proximal from the tip 44. The slots 48 expose a lumen of the needle 40 to the exterior of the needle 40. A proximal end of a stylet 50 is connected to the stylet handle component 26. A distal end of the stylet 50 is split into multiple flexible prongs 52 at a distal section. The distal ends of the prongs 52 are connected to each other at a distal plug 54. The stylet 50 and the prongs 52 may be manufactured by laser cutting a hypotube or rod.

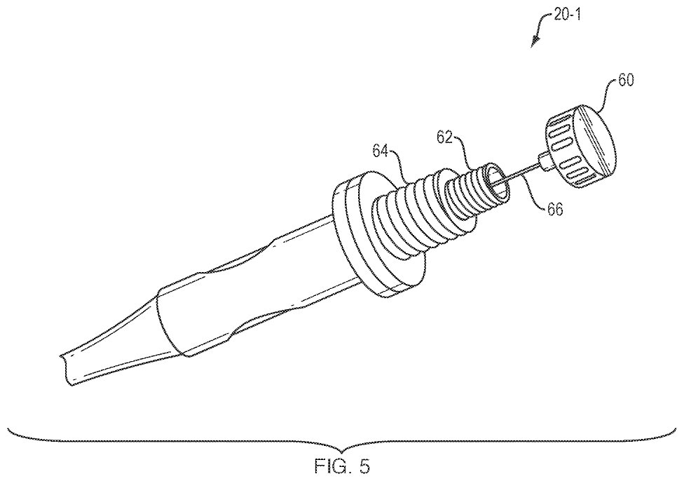

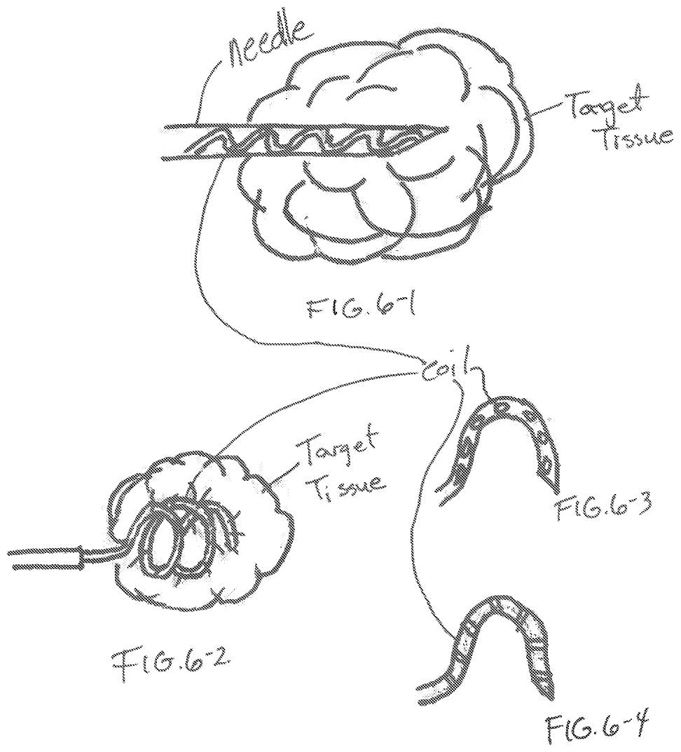

Once the needle 40 has been positioned adjacent to previously identified target tissue with the help of the endoscope 12, the actuator component 24 is advanced distally causing the needle 40 to penetrate the target tissue 70 (FIG. 3). Next, the user applies a distal force to the stylet handle component 26. When pressure/force is applied to the stylet handle component 26, the distal plug 54 makes contact with the distal end of the lumen of the needle 40 or inside of the tip 44. This causes the prongs 52 of the stylet 50 bend outward from a longitudinal axis of the needle 40, thus passing through the slots 48. The prongs 52 protruding from the slots 48 penetrate or apply pressure to the surrounding target tissue 70 (FIG. 4). Then the needle 40, if desired, could be rotated, allowing the prongs 52 to cut the tissue and subsequently create more or larger pockets within the target tissue for fluid, such as a medicant, to flow. Once the distal force is released, the prongs 52 retract within the needle 40 and return to the predeployed position. Then fluid could be injected through the needle lumen and out of the slots 48 at the tip of the needle 40. The stylet 52 would be thin enough that there would be room within the needle lumen for fluid to flow.

In one embodiment, the prongs 52 have edges sharp enough to cut tissue. Thus, when the needle 40 is rotated, the sharp edges of any protruding prongs 52 will cut surrounding tissue.

In one embodiment, the distal plug 54 and the tip 44 are configured such that when the distal plug 54 is forced against the interior of the tip 44, the distal plug 54 rotates into proper alignment so that the prongs 52 rotate to align with the slots 48. This is considered a clocking mechanism. Other clocking mechanisms can be used.

In one embodiment, as shown in FIG. 5, a stylet handle component 20-1 includes a screw/twist knob 60 that mates to a luer 62 at the proximal end of an actuator handle component 64. If the stylet twist knob 60 is not connected to the luer 62, but simply rests on top of it, then a distal end of a stylet 66 will sit in the middle of a bevel cut of the distal end of a needle. The prongs 52 will not be forced to expand out of the needle slots 48. By connecting the stylet knob 60 and luer 62, the stylet 66 compresses against the tip 44 of the needle, thus causing the prongs 52 to protrude through the slots 48. How far the prongs 52 protrude depends on how far the knob 60 is twisted onto the luer 62.

In one embodiment, a stylet knob includes a counterbore with a spring. When the knob rests on the luer at the proximal end of the device, a distal tip of the stylet attached to the knob lies within the distal end of the needle. When the spring is compressed by a distal force applied by a user to the stylet knob, the distal force causes the prongs of the stylet to extend out of slots at the distal end of the needle.

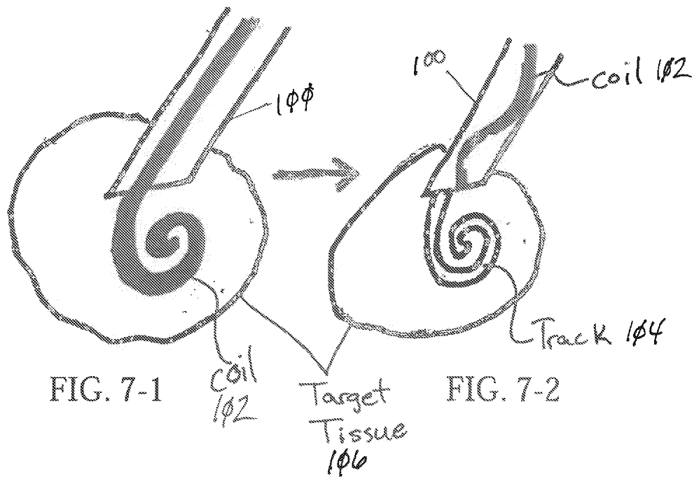

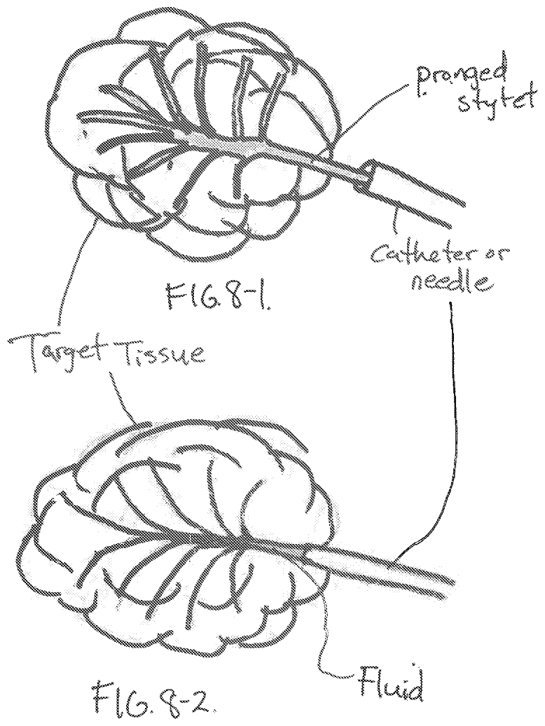

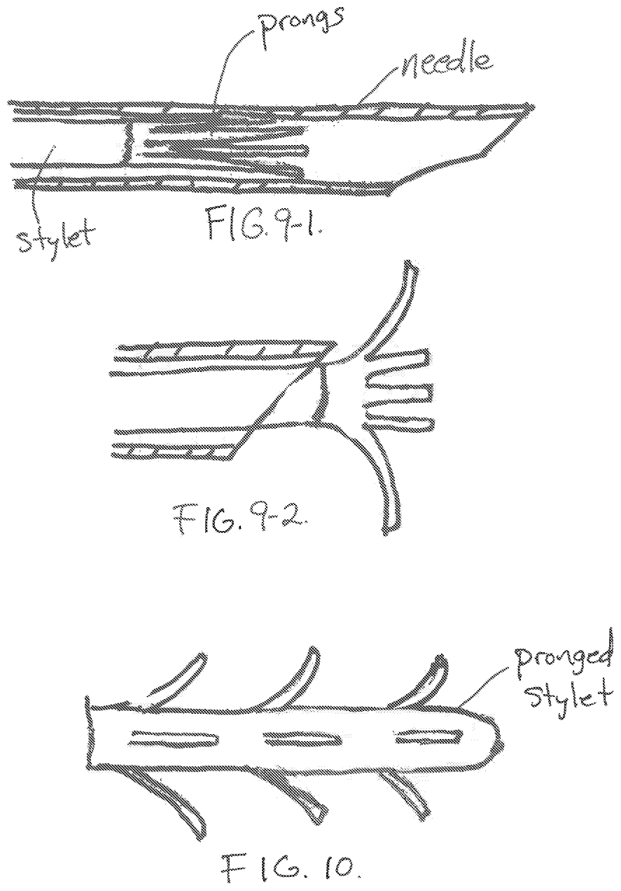

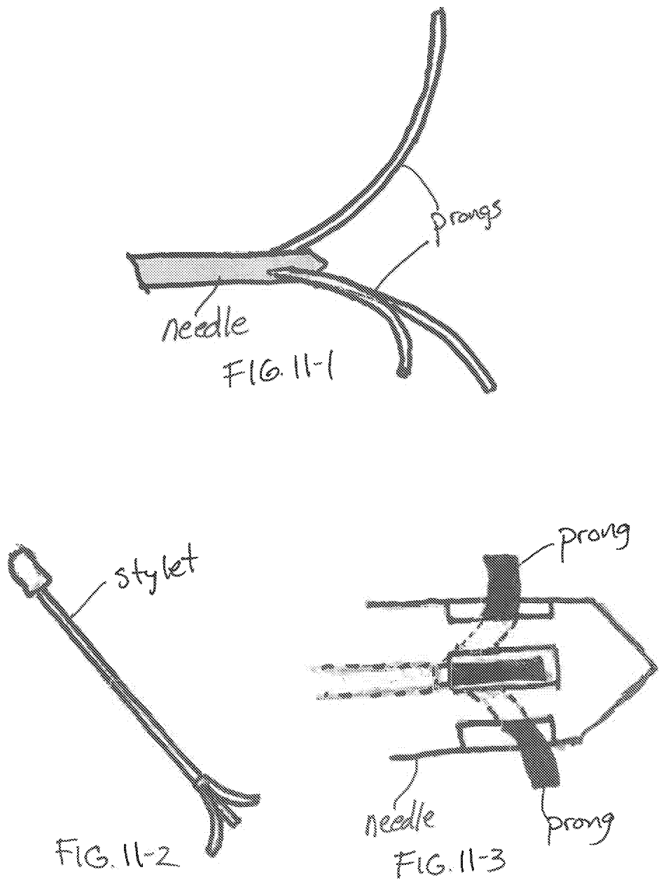

In one embodiment, as shown in FIGS. 7-1 and 7-2, a needle 100 attached to a handle (not shown) is advanced into target tissue 106 is a guide device, such as a bronchoscope. A coil 102 is initially located within a lumen of the needle 100 and advances with the needle 100. The coil 102 is attached to a component of the handle distinct from the handle component that connects to the needle 100. Once the needle 100 has been advanced into the target tissue 106 as identified by some imaging system, the coil 102 extended to beyond the distal end of the needle 100. As the coil 102 extends beyond the distal end of the needle 100, the coil 102 changes from a shape that conforms to the needle 100 to its predefined shape, which is a coil or helical configuration. The coil 102 may be formed of a shape-memory metal. The coil 102 is then retracted back into the needle 100. After coil retraction has occurred, a treatment solution device is attached to the handle and the medicant solution device forces a treatment solution through the handle and into the needle 100 and out the distal end of the needle 100 into the target tissue 106.

In another embodiment, the steps of deploying the coil 102 and retracting the coil 102 into the target tissue 106 is repeated one or more times before the deployment of the treatment solution with the needle 100 being at a different position within the target tissue 106. The device shown in FIGS. 7-1 and 7-2 creates one or more tracks within the target tissue that allows for flow of the treatment solution.

The description is merely exemplary in nature and variations that do not depart from the gist of the embodiments are intended to be within the scope of the invention. Such variations are not to be regarded as a departure from the spirit and scope of the invention.

What is claimed is:

1. A medical device adapted to deliver a medicant into a target tissue, the medical device comprising:
a first tissue penetrating device;
a second tissue penetrating device configured to extend from the first tissue penetrating device to form a track within the target tissue, in an extended state the second tissue penetrating device forms a plurality of prongs extending from slots in the first tissue penetrating device, in a retracted state the second tissue penetrating device conforms to the first tissue penetrating device; and
a handle comprising:
a first component connected to the first tissue penetrating device;
a second component connected to the second tissue penetrating device; and
a fluid delivery component configured to allow fluid to pass through the first tissue penetrating device and into the track formed in the target tissue by the second tissue penetrating device,
wherein when the second component is at a first position relative to the first component, the second tissue penetrating device is in an undeployed configuration,
wherein when the second component is at a second position relative to the first component, the second tissue penetrating device is in a deployed configuration.

2. The medical device of claim 1, wherein the first and second tissue penetrating devices are configured to repeatedly allow the second tissue penetrating device to extend from and retract into the first tissue penetrating device.

3. The medical device of claim 1, wherein each prong of the plurality of prongs include sharp edges to facilitate cutting of tissue upon extension out of the slots of the first tissue penetrating device.

4. The medical device of claim 1, wherein the plurality of prongs are formed in a distal section of the second tissue penetrating device.

5. The medical device of claim 4, wherein the distal section of the second tissue penetrating device is split into multiple sections to form the plurality of prongs.

6. The medical device of claim 4, wherein a distal end of each prong of the plurality of prongs is coupled together by a distal plug.

7. The medical device of claim 6, wherein the distal plug includes a clocking mechanism to align the plurality of prongs with the slots.

8. The medical device of claim 7, wherein the clocking mechanism includes an outer surface of the distal plug that interacts with an interior of a tip of the first tissue penetrating device to align the plurality of prongs with the slots.

9. A method comprising:
a) inserting a hollow needle into a target;
b) deploying a plurality of prongs from within the hollow needle and generating one or more pockets in a target tissue;
c) retracting the plurality of prongs back into the hollow needle; and
d) advancing medicant solution through the hollow needle, around the retracted plurality of prongs, to the one or more pockets in the target created by deploying the plurality of prongs into the target tissue.

10. The method of claim 9, further comprising:
before d) performing the following one or more times:
at least one of retracting or advancing the hollow needle into a different location of the target; and
repeating b) and c).

11. A medical device adapted to deliver a medicant into a target tissue, the medical device comprising:
a needle including a lumen extending from a proximal opening to a distal end adjacent a tip of the needle;
a stylet configured to be delivered to a target tissue through the lumen of the needle, the stylet including a plurality of prongs configured to extend out of slots proximal from the tip of the needle upon extension of the stylet through the lumen; and
a handle comprising:
a first component connected to the needle;
a second component connected to the stylet; and
a fluid delivery component configured to allow fluid to pass through the needle and into pockets formed in the target tissue by the plurality of prongs of the stylet.

12. The medical device of claim 11, wherein when the stylet is at a first position relative to the needle, the plurality of prongs of the stylet are in an undeployed configuration within the lumen; and
wherein when the stylet is at a second position relative to the needle, the plurality of prongs of the stylet are in a deployed configuration extending out through the slots in the needle.

13. The medical device of claim 11, wherein each prong of the plurality of prongs include sharp edges to facilitate cutting of tissue upon extension out of the slots of the needle.

14. The medical device of claim 11, wherein the plurality of prongs are formed in a distal section of the stylet.

15. The medical device of claim 14, wherein the distal section of the stylet is split into multiple sections to form the plurality of prongs.

16. The medical device of claim 14, wherein the distal end of each prong of the plurality of prongs is coupled together by a distal plug.

17. The medical device of claim 16, wherein the distal plug includes a clocking mechanism to align the plurality of prongs with the slots.

18. The medical device of claim 17, wherein the clocking mechanism includes an outer surface of the distal plug that interacts with an interior of the tip of the needle to align the plurality of prongs with the slots.

19. The medical device of claim 11, wherein each prong of the plurality of prongs includes a distal end adapted to extend out of one of the slots in the needle upon extension of the stylet through the lumen.
